(19) United States
(12) Patent Application Publication
(10) Pub. No.: US 2023/0369821 A1
(43) Pub. Date: Nov. 16, 2023

(54) AUTOMATIC LASER SETTING ADJUSTMENT

(71) Applicant: GYRUS ACMI, INC. D/B/A OLYMPUS SURGICAL TECHNOLOGIES AMERICA, Westborough, MA (US)

(72) Inventors: Kurt G. Shelton, Bedfird, MA (US); Kester Julian Batchelor, Mound, MN (US); Tyrel J. Waagen, Douglas, MA (US)

(21) Appl. No.: 18/297,719

(22) Filed: Apr. 10, 2023

Related U.S. Application Data
(60) Provisional application No. 63/364,460, filed on May 10, 2022.

(51) Int. Cl. H01S 3/102; H01S 3/106; H01S 3/10; H01S 3/131; H01S 3/13

(52) U.S. Cl. CPC H01S 3/102; H01S 3/10069; H01S 3/106; H01S 3/1305; H01S 3/131

(57) ABSTRACT

Disclosed are system and methods for a laser system comprising at least one laser, a processor, and a user interface. The processor can receive a range for at least one setting of the at least one laser that the at least one laser can operate within. The processor can further determine a proposed updated value for the at least one setting and determine that the proposed updated value is within the range. An indication of the proposed updated value and an option for a user to accept or reject the proposed updated value can be provided on a user interface. Responsive to an acceptance the at least one setting can be adjusted based on the proposed updated value. Responsive to no rejection within a predetermined period of time, the processor can cause the at least one setting to be adjusted or cancel adjustment of the at least one setting.

### CLAIM OF PRIORITY

This application claims the benefit of priority to U.S. Provisional Pat. Application No. 63/364,460 filed May 10, 2022, the contents of which are incorporated herein by reference.

### TECHNICAL FIELD

The present disclosure relates to laser settings during medical procedures using laser radiation.

### BACKGROUND

Laser systems are used during both therapeutic and diagnostic medical procedures, such as in systems in which a laser fiber is used as a part of an in-vivo endoscopic system. During surgical laser procedures, such as laser lithotripsy, lasers are used to ablate or cut tissue or reduce stones, such as kidney stones or gall stones, into small fragments that can either be passed from the body naturally or actively removed using retrieval devices or by flushing using a solution such as saline.

### SUMMARY

Depending on what type of medical procedure is being performed, the intensity of the laser or lasers being used for the procedure may need to be changed or adjusted. The amount of time that it takes to reduce a stone or treat tissue can be decreased by using laser settings that are optimized for the specific type of stone or tissue being targeted. For example, during ablation of a “calculus” (a stone) such as a kidney stone or a gall stone, the composition of the stone may change from a harder substance to a softer substance or vice-versa. For example, a stone may be comprised partially of Calcium Oxalate and Calcium Phosphate such that it has a hard “shell” and a softer “core”. In another example in which a patient has multiple kidney stones, some stones can be formed from Calcium Oxalate, others from Calcium Phosphate, and still others from Uric Acid. Each stone type can require a different laser intensity to efficiently break up the stone (e.g., to reduce the size of the stone and/or turn the stone to dust) so that it can pass through the body or otherwise be removed. Other procedures may involve a physician using laser energy on a portion of tissue, such as bladder tissue, which may require a different laser type and/or laser intensity than used to break up or reduce a stone.

The present inventors have recognized, among other things, a need for a laser system with the ability to receive or determine a range for a setting of a laser and during a medical procedure, determine a need for an update or adjustment to the settings based on, for example, a condition experienced or encountered during the procedure. A laser system can include a processor or processing circuitry that can receive a range for at least one setting for a laser included in the laser system that the laser can operate within. The processing circuitry can be included as a part of a surgical laser system or can be included as a component of a computer or machine that the laser is coupled to or connected to. The at least one setting can include a wavelength or intensity of the laser radiation, a power setting, a spot size of the laser, a pulse width of the laser, a duty cycle of the laser pulses, or any available setting or parameter of the laser. The range for the setting can be based on a type of procedure being performed, and can be determined, entered, or retrieved before the procedure. For example, the range for the setting or settings can be entered by a physician or user on a Graphical User Interface (GUI) included as a part of the laser system or connected to the laser system, and/or can be retrieved from a patient file retrievable by the processing circuitry from a database or server connected to the laser system (or a machine to which the laser system is connected to).

The laser system can receive a confirmation for the user or otherwise determine that the laser can operate automatically within the range. For example, that the laser can be automatically adjusted to different wavelengths within a predetermined range of wavelengths set by, for example, the user. The system can determine a need for a change or adjustment to the setting and indicate a proposed updated value for the setting. The proposed updated value can be a different value within the range from the current value. For example, the range of the laser wavelength may be from 700 nanometers (nm) to 900 nm, and the current value of the wavelength may be set to 750 nm. The system can determine that the wavelength should be updated to 900 nm, and provide an indication on the GUI, such as through a message that pops up on the GUI. The system can include, as a part of the indication or separate from the indication an option for a user to accept or reject the proposed updated value. For example, the message that pops up providing the indication may include an accept button or a reject button. Responsive to an acceptance of the proposed updated value or no rejection of the proposed updated value by the user within a predetermined duration (e.g., a set period of time such as 10 seconds), the laser system can adjust the laser setting based on the proposed updated value.

Thus, the system can include one or more of the following features to be discussed in more detail below:
i) “Laser Interlocking” in which the laser emission can be temporarily stopped or disabled while the laser setting(s) are updated and resumed after receiving a command from the laser system and/or acceptance of the new setting by the user;
ii) Automatic Adjustment of the laser settings in which one or more settings of the laser can be adjusted within the range based on composition of the target, a change in composition of the target (e.g., based on a spectroscopic analysis of the target), or any other relevant parameter;
iii) Prompted laser settings adjustment in which the laser system can propose or recommend adjustment of one or more laser settings to a user and prompt the user to accept or reject the new, updated settings; and/or
iv) Display Target Analysis information in which information about the target (or changes to the target) can be displayed on the GUI and updated in real-time during the procedure.

In the automatic adjustment mode, the system may provide the user with an indication or notification such as via a pop-up screen on the GUI, of a new laser setting that the physician can accept or cancel the adjustment. During this time the laser system may be locked or disabled using laser interlocking, such that the laser cannot pulse or emit. If the user accepts the change the laser can remain locked until the adjustment is made, and then laser emission can resume under the new setting(s). If the user cancels or rejects the adjustment, laser emission can resume under the current laser setting(s). If, however, the user does not respond to the indication (e.g., the user does not affirmatively accept the adjustment, but does not reject the adjustment within a period of time) the system can automatically adjust the setting and resume laser emission without additional user interaction as long as the user continues to engage laser emission (e.g., as long as a footswitch continues to be depressed a button or trigger is pressed, or the like). Accordingly, the automatic adjustment mode provides an approach that allows the system to automatically adjust the laser settings if the proposed value is within the predetermined, safe range and the user does not object to the adjustment.

Conversely, in the prompted adjustment mode, if the user does not respond to or ignores the indication, but does not affirmatively cancel the adjustment, no change to the current laser setting(s) will be made. In sum, in the automatic adjustment mode, the laser settings will change when a user either affirmatively accepts the new settings or fails to reject the new settings within a period of time (e.g., two seconds, or any appropriate or desired time frame). In the prompted adjustment mode, the laser settings will only change when the user affirmatively accepts the changes. The laser interlocking and/or the target information can occur in either the automatic adjustment mode or the prompted adjustment mode.

The terms “physician” and “user” are used interchangeably herein and are understood to include anyone who may be operating the laser system. It is also understood that the term “laser system” and “the system” are also used interchangeably.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a system for automatic laser adjustment.

FIG. 2 illustrates an example of a method for automatic adjustment of a laser system.

FIG. 3 is a flowchart for operation of a laser adjustment system.

FIG. 4 is a block diagram illustrating an example of a machine upon which one or more embodiments may be implemented.

FIG. 5 illustrates a schematic diagram of an exemplary computer-based clinical decision support system (CDSS).

### DETAILED DESCRIPTION

Lasers are used in medical procedures to, among other things, reduce stones such as kidney stones or gallstones into smaller fragments that can either be passed naturally through the body or actively removed. The amount of time required to reduce a stone varies depending on factors such as the material composition of the stone, and the amount of time required to reduce or break apart the stone can be decreased by adjusting the laser settings to be optimized for the specific type of stone being targeted. For example, a laser emitting a higher pulse energy and a lower frequency may be appropriate for a stone composed or made up predominately of Calcium Phosphate, whereas a laser emitting a lower pulse energy and a higher frequency may be appropriate for a stone composed or made up predominately of Calcium Oxalate.

Further, the composition of a stone may be heterogeneous such that the outer portion (e.g., an outer shell) of the stone may have a different material composition or a different density than the inner portion or inner volume. In such an example, to most efficiently reduce the stone, different laser settings may be optimal when ablating or treating the outer shell than when treating the inner volume. Thus, a system capable of identifying the composition of the stone, and when the composition of the stone changes from one material to another or from one density to another, such as changing from Calcium Oxalate to Calcium Phosphate, and automatically adjust the laser settings is desired. This can ensure that a physician always delivers the optimal laser energy, both intensity and frequency, while reducing a heterogenous stone.

In an example, the system can notify or prompt the physician, such as through a Graphical User Interface (GUI), of an adjustment to the laser settings. For example, when the system detects a change in the composition of the stone, the system can notify the physician on the display that the composition of the stone has changed and that an adjustment to the laser settings should be made. The notification can be made as a recommendation to the physician to change from a current laser setting or settings to a new laser setting and require an input from the physician to accept or approve the changed setting. Further, the information regarding the stone or any other target can be displayed on the same monitor or display showing the endoscopic video of the laser procedure to minimize or eliminate the need to redirect the focus of the physician from the endoscopic view to a different monitor.

Another medical procedure in which the laser system can be used is a procedure in which soft tissue (as opposed to a stone) is being treated, such as during Benign Prostatic Hyperplasia (BPH) treatment. For example, BPH treatment can involve using a laser to incise an adenoma while avoiding the prostatic capsule so as to not perforate the prostatic capsule. The system can automatically measure the type of tissue being targeted by the surgical fiber and disable the laser emission while the capsular tissue is measured. In another example, instead of a stone or an enlarged prostate, the target can be a tumor, such as a cancerous tumor on an organ such as the bladder. In such an example, the system can automatically disable the laser emission when non-cancerous tissue is detected or being treated.

Whether the laser fiber is being used to treat soft or hard tissue, when automatically stopping laser emission and/or changing a laser setting optimized for a particular tissue it is desirable to: 1) change the laser setting to a setting determined (e.g., by the physician) to be safe for the specific patient being treated; and 2) inform the physician when the system determines the target or tissue has changed, and what it has changed to. The change in the target or tissue can be determined by, for example, receiving information from a spectroscopy system that is connected to or communicatively coupled to the laser system.

Such a system represents an improvement over existing medical laser systems that do not include an automatic interlocking system and/or an automatic laser adjustment system that responds to a changing target. Laser procedures performed with existing systems experience less efficient treatment procedures, longer treatment durations, and less efficient treatment outcomes. These efficiency problems are greater when dealing with targets or tissues, the treatment of which, strongly depend on the skill and experience level of the physician. The presently disclosed laser system provides the physician with the opportunity to override the determination and/or recommendation of the system to make changes to one or more settings of the laser, and/or to constrain the system to make changes to the laser setting within a defined range determined by the physician to be safe for the specific patient. The physician can require that a confirmation by the physician is provided before any change to the laser settings is made. Further, the system can be set or configured to operate in an automatic or semi-automatic mode for a predetermined duration or a predetermined limit, such as for a time limit, a total energy delivered limit, the duration of a single procedure, or any other duration or limit desired or appropriate.

In an example, the laser system, even when operating in an automatic mode, can provide an alert or notification to the physician that one or more settings will change and what the setting or settings will be changed to, and provide the physician the opportunity to cease the laser emission, such as by releasing a footswitch or foot pedal when the physician disagrees with the new setting or wants to delay implementation of the new setting. In another example, in a semi-automatic mode, prior to a setting change, the system can provide an indication on the GUI requesting approval to change to the new setting. In such an example, the change to the new setting can be implemented upon approval by the physician or by the system receiving from the physician no rejection of the new setting within a predetermined duration or period of time.

Example implementations of the system can include:

Interlocking the laser emission (“Laser Interlocking”). This can include temporarily ceasing or stopping and then resuming a laser emission after receiving a command, such as from a spectroscopy system. For example, a 1940 nm laser emission can be ceased and then resumed within 50 milliseconds (ms) after receiving the command. In such an example, the laser emission can be resumed so long as the physician continues to depress the footswitch, without requiring other physician or user interaction with the system. The GUI can be capable of indicating to the physician when the laser is interlocked such as through a message or graphic on the GUI.

Automatic laser settings adjustment (“Automatic Adjustment”) mode. This can include automatically adjusting laser settings based on spectral signature information about a target received from a spectroscopy system. Such spectral signature information about the target can be used by a processor to determine an updated laser setting or one or more updated ranges of one or more laser settings. In various embodiments, prior to and/or during the medical procedure, the system processor can require the physician to provide, e.g., via the GUI, a range of settings that the laser system can be permitted to automatically adjust within. The system processor can (i) compare the updated setting(s) with the value(s) that the laser system is permitted to automatically adjust within and (ii) confirm that the laser system can operate in an automatic adjustment mode within those physician-specified or physician-approved ranges. Once confirmed, the laser system can be automatically adjusted based on the determined updated setting(s). In some embodiments, information about the updated laser setting or range can be presented to the physician, for example, by the GUI such as on a pop-up or pop-out screen on the GUI. The system processor can also provide the physician an opportunity to approve or cancel the adjustment (e.g., by indicating to the physician that the new setting will be implemented and requesting approval by the physician before performing such update). For example, the pop-up screen may include one or more buttons, such as an accept or deny button, a yes or no button, or any similar button that will allow the physician to approve or cancel the adjustment. In the automatic adjustment mode, when the physician ignores the popup screen, does not cancel or exit out of the popup screen, etc., for a predetermined period of time or duration (e.g., 10 seconds) the system can automatically adjust to the new or updated proposed laser setting(s). The system can then temporarily interlock the laser emission and adjust or change the existing laser settings to the new settings and then resume laser emission without additional interaction as long as the physician continues to depress the footswitch.

Prompted laser settings adjustment, (“Prompted Adjustment”) mode. This can include adjusting laser settings to those received from a processor, such as based on target spectral signature information from a spectroscopy system. The processor can trigger displaying the adjusted settings on the GUI. In such an example, the physician can be requested to provide a range of settings within which the laser system can propose new settings. The physician can confirm that the laser system can operate in a Prompted Adjustment mode within the range or ranges specified by the physician. The system can then propose a new setting for the laser, such as based on updated spectral signature information about the target received from the spectroscopy system. The system can require the physician to affirmatively select the proposed new laser setting to apply and put the proposed new laser setting into effect. For example, this can involve the physician approving the proposed laser setting on the GUI. The proposed setting may be indicated to the physician such as through a popup screen or box on the GUI. In an example, when the physician ignores (for a predetermined period of time or duration, such as 10 seconds) or cancels the popup screen, no change to the currently emitting laser settings is made in the prompted adjustment mode. When the physician affirmatively selects to apply (or does not reject) the proposed settings, the system can temporarily interlock the laser emission, adjust the laser settings to the new settings, and resuming laser emission without additional user interaction so long as the footswitch continues to be depressed. Accordingly, the proposed adjustment mode provides an approach that allows the system to adjust the laser settings only if the proposed value is within the predetermined, safe range and the user has affirmatively accept the proposed adjustment.

Display Target Analysis information, (“Target Information”). This can include using the GUI to display information received about the target from a spectroscopy system and/or processor (e.g., on Treatment and Emissions screens). For example, the GUI can display estimated or measured composition of a target stone, or type of soft tissue, which can be updated as changes in the target or type of tissue are detected.

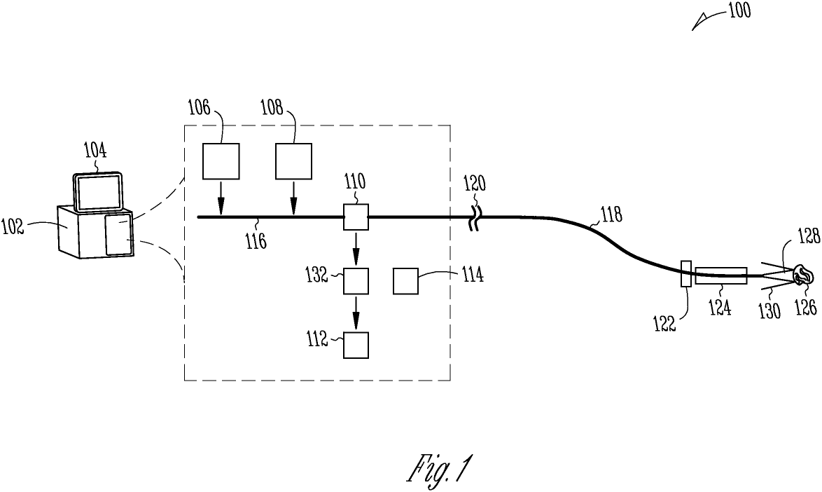

FIG. 1 illustrates an example of portions of a system 100 for automatic laser adjustment. The system 100 can include a surgical laser 102. The system may include a graphical user interface (GUI) 104. The GUI 104 may include a touchscreen or other input mechanism (e.g., a button, switch, or other similar actuation member on the handle of the endoscope) configured to operate, control, or the like, the surgical laser 102. The surgical laser 102 can include one or more laser sources configured to emit laser radiation. As shown in the dashed box in the example of FIG. 1, the laser sources can include an ablation laser 106 and/or a probe laser 108. The ablation laser 106 can emit infrared radiation while the probe laser 108 can emit an aiming beam or an illumination beam of visible light (such as from a light emitting diode (LED) to show where the tip of the scope (and therefore where the ablation energy from the ablation laser 106) is aimed or can be used to illuminate a target 126. The target 126 can be a piece of tissue, debris, or an object, such as a kidney stone which is to be ablated, or the like. Thus, the emitted light 128 can be emitted from the ablation laser 106 or the probe laser 108 independently of each other or in conjunction with each other, meaning that the emitted light 128 emitted from the surgical laser can be visible, invisible, or both (e.g., a combination of infrared light and visible light).

The emitted light 128 from the laser sources 106, 108, can be emitted through an optical fiber 116 such as can be connected to a surgical fiber 118 via an optical connector 120. In an example, the structure of the surgical fiber 118 can be the same or different from that of the optical fiber 116. The surgical fiber 118 can be located wholly or partially outside the surgical laser 102. The emitted light 128 can thus be emitted from the laser sources 106, 108, through the optical fiber 116, the optical connector 120, and the surgical fiber 118, to a distal end of the surgical fiber 118 which can be inserted into a scope 124, such as an endoscope, a ureteroscope, laryngoscope, or the like. In an example, at least a portion of the emitted light 128 emitted from the distal end of the surgical fiber 118 and the scope 124 can be reflected off of a target 126 (reflected light 130) through a medium between the tip of the scope 124 and the target 126.

The surgical laser 102 can further include or couple to an optical component such as optical splitter 110, configured to collect at least a portion of the reflected light 130 passing through the aperture of the surgical fiber 118. In an example, the optical splitter 110 can be replaced with a dedicated fiber configured to collect at least a portion of the reflected light 130. In an example, the portion of reflected light 130 collected by the optical splitter 110 or dedicated fiber can be sent to a processor 112 in connection with the surgical laser 102. A light detector 132 (e.g., a spectrometer) can be located between the optical splitter 110 and the processor 112, so that spectral analysis of the of the reflected light 130 can be performed in order to determine one or more characteristics of the target 126 and a determination can be made (e.g., by the processor 112) whether one or more laser settings should be adjusted. Before a medical procedure, a range for one or more settings for the laser sources 106, 108 can be provided to or received by the processor 112. In an example, a setting can be an intensity of laser light or laser radiation or an amount of energy to be emitted from one of the laser sources 106, 108, and the range can be intensity values (e.g., an upper-threshold value or upper limit and a lower-threshold value or lower limit, and the values therebetween) that the ablation laser 106 and/or the probe laser 108 can operate within. In another example, the setting can be a wavelength of the laser and the range can be an upper-threshold wavelength value, a lower-threshold wavelength value, and the values therebetween. In such an example, changing the wavelength value may include switching to a second laser diode with a higher or lower wavelength than a currently active or in use laser diode as adjusting the wavelength of a single diode is generally not something that can be done easily. In yet another example, a setting can be a pulse width or duty cycle of the emitted laser light.

In an example, the processor 112 can receive a confirmation such as from a physician, that the laser can operate automatically within the range. At least one of the range or a limit of the range can be determined based on at least one of: a type of anatomy that the scope 124 is in proximity to (e.g., whether the scope is in a kidney or outside a kidney, a characteristic of the anatomy that the scope 124 and/or the surgical fiber 118 is in proximity to (e.g., whether the anatomy includes healthy tissue or a target to be ablated), a type of laser fiber included in the surgical laser 102 or to be used during the medical procedure, or a type of medical procedure for which the system 100 is being used.

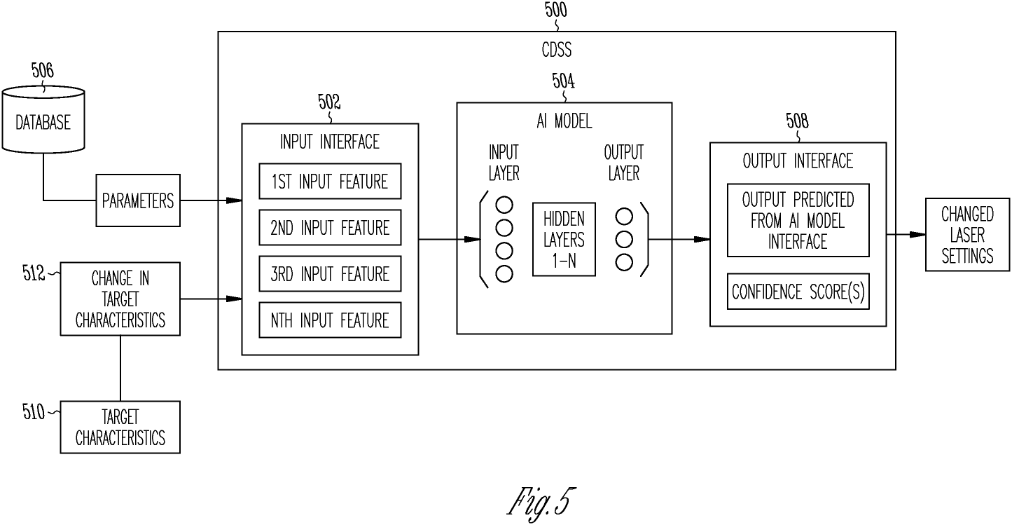

In an example, the portion of anatomy can be determined based on any suitable image recognition technique performed on an image from an imaging device connected to the scope 124. For example, a camera can be connected to the scope 124 and capture an image (e.g., a still image or a video image) and the system 100 can perform the image recognition technique to determine what portion of a patient’s anatomy the scope 124 (or a portion of the scope 124 such as the tip of the scope 124) is currently in or near. The type of medical procedure may be entered by the physician prior to the procedure, such as on the graphical user interface 104 connected to the surgical laser 102. Alternatively, the processor 112 can be communicatively coupled to a database containing patient information (such as the databases discussed below for FIG. 5 and FIG. 6), and the type of medical procedure can be determined from the patient information in the database.

The processor 112 and/or light detector 132 can analyze the portion of the reflected light 130 collected by the optical splitter 110 (or receive an analysis by a spectrometer connected or coupled to the processor 112 and/or the light detector 132), and determine, based on the analysis of the reflected light 130, a proposed updated value for the laser setting. Additionally or alternatively, the determination can be made based on an environmental condition measured by a sensor coupled to the processor 112 such as a temperature value or range at the tip of the scope 124 measured by a temperature sensor coupled to or included on the scope 124. Additionally, or alternatively, the environmental condition can be a pressure value or range, such as the pressure in a medium in which the scope 124 is located. In another example, the determination can be made based on a characteristic of the target 126. For example, the processor 112 and/or the light detector 132 can analyze the reflected light 130 from the target 126 to determine a characteristic of the target 126 at a first time. The characteristic can be a composition (a material composition) or a size of the target 126 at the first time. The processor 112 can then analyze the reflected light 130 from the target 126 at a second time during the procedure and determine a change in the characteristic of the target 126 at the second time by comparing the characteristic of the target 126 at the first time and the characteristic of the target 126 at the second time. For example, when the target is a stone or other calculus, at the second time the composition of the target 126 may change such as from a harder material to a softer material, or the target 126 may be substantially smaller at the second time than at the first time, such that less laser energy is needed to ablate or break up the target 126. Based on the determination of the change in the characteristic of the target at the second time, the system can determine the proposed updated value. Additionally, or alternatively, the proposed updated value can be based at least in part on a position of one of the laser fibers such as a distance of the ablation laser 106 and/or the probe laser 108 and/or the tip of the scope 124 from the target 126. For example, the closer the laser fibers or the tip of the scope 124 is to the target 126 the less energy may need to be emitted, and thus the intensity of the laser emission can be lowered and/or the duration of the laser pulse can be adjusted.

The proposed updated value can be indicated, and an option to accept or reject the proposed updated value can be provided, on the graphical user interface 104. For example, when the processor 112 determines a need for a change to the setting(s), an indication or notification, such as in the form of a pop-up box or menu, can be sent to the graphical user interface 104 informing the physician that an updated setting is recommended, or an updated setting will be implemented. The notification can include a value of the updated setting(s), and while the notification is displayed on the graphical user interface 104, laser emission can continue or, in some embodiments, be locked, disabled, terminated, cut-off, or otherwise prevented (via the “Laser Interlocking” discussed above). In an example, the notification can include a button, link, or the like on the pop-up screen or somewhere else on the graphical user interface 104 for the physician to accept or reject the proposed updated setting. For example, the pop-up screen with the proposed change may include an “accept” button and/or a “reject” button, that a user can click on. In another example, the acceptance or rejection of the updated value can be initiated via a voice command, or activation of an actuation member. The actuation member can include a button or a switch on the scope 124 or a handpiece of (or connected to) the scope 124, or a footswitch or foot pedal connected to the system 100, such as connected to the surgical laser 102 and/or the scope 124.

In an example, the system 100 can include a feedback mechanism to provide an additional indication or notification which can accompany or be included with the notification discussed above. The additional indication can notify or warn the physician that the proposed updated value has been determined. The additional indication can be through a haptic feedback mechanism, such as vibration of the handpiece of the scope 124, an audible feedback mechanism, such as a beep or chime through a speaker or similar output device, or an illumination feedback mechanism, such as changing a characteristic of a user-visible targeting illumination beam emitted toward the target. For example, when the processor 112 determines a need for an updated setting, the processor 112 can cause a chime or beep to be emitted through a speaker included in the graphical user interface 104, can cause the handpiece of the scope 124 to vibrate, can cause a button on the handpiece of the scope 124 to illuminate or light up and/or can cause a visible light beam emitted through the scope 124 to blink, change color, or the like.

The additional notification can also function as a warning that a setting of the one or more settings is within a threshold amount of an upper limit of the range. For example, the additional notification can inform the user when the intensity of radiation, light, or signal emitted from the laser sources 106, 108 is near the upper limit of the range established before the medical procedure. This additional notification or warning can be especially useful when the system changes the settings automatically within the range without interaction or approval from the user (e.g., in a fully automatic system) so that the user is aware of when the setting(s) are close to the upper limit of the range and can make any adjustments desired or appropriate.

The surgical laser 102 may optionally or additionally include a controller 114 communicatively coupled to the processor 112. In response to the acceptance of the proposed updated value or values, the controller 114 can adjust the current setting(s) of the laser to the updated setting(s). In an example, the changed setting can include causing a change in the intensity of the emitted laser radiation/light 128 (e.g., stopping, reducing, lowering, increasing, raising or the like) based on one or more factors such as distance from the target 126, a change in composition of the target 126 or any relevant factor warranting an adjustment to the intensity of the emitted laser radiation or light 128. In another example, the adjustment of the setting can include a change in one or more other parameters (e.g., duty cycle, pulse width, or the like) of the light or radiation emitted from one of the lasers. Additionally, or alternatively, the controller 114 can cause a surgical fiber actuator 122 configured to be connected to the surgical fiber 118 to adjust a location of at least a portion of the surgical fiber 118. For example, the surgical fiber actuator 122 can cause the surgical fiber 118, such as the portion of the surgical fiber 118 connected to the scope 124 to change its location (e.g., move closer to or away from the target).

More than one setting can be adjusted, and the adjustments can be applied independently or in conjunction with each other as desired or appropriate. The processor 112 and/or the controller 114 can be configured to automatically select and cause one or more suitable adjustments to one or more settings to be applied. By configuring the processor 112 and/or the controller 114 to automatically select and cause a setting adjustment to be applied based on the analysis of the reflected light 130, the system can provide increased ablation or surgical efficiency as the processor 112 and/or the controller 114 can cause the adjustment to be applied faster than a human can react to changing conditions during the medical procedure, resulting in more efficient and effective laser procedures.

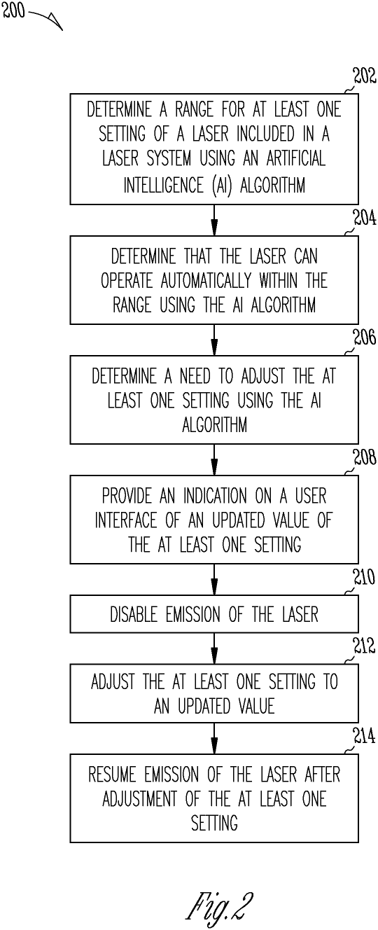

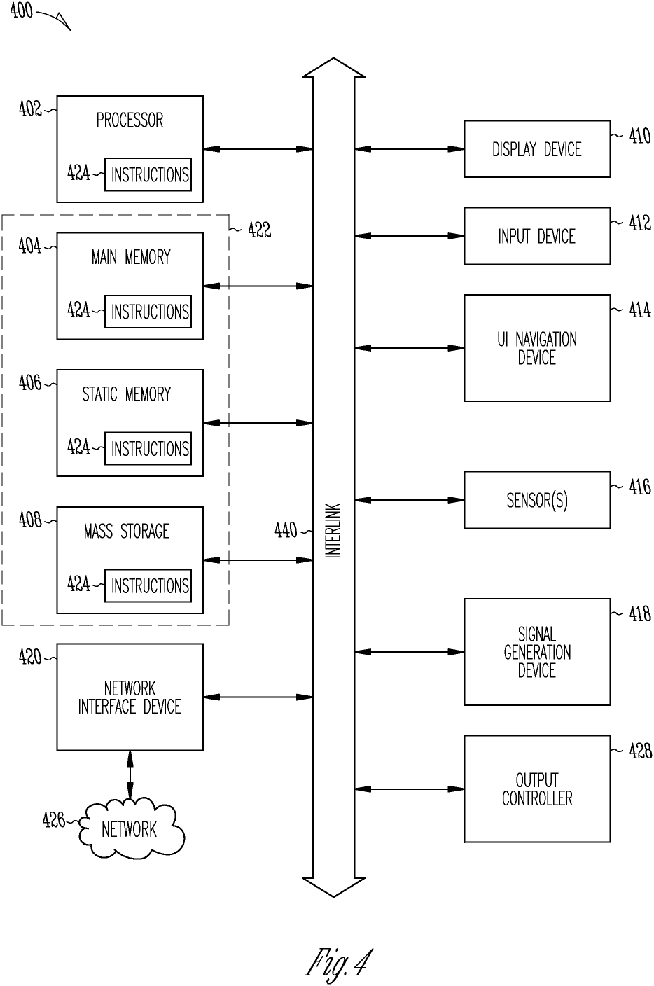

FIG. 2 illustrates an example method 200 for automatic adjustment of a laser system. The method 200 can include or comprise a number of Operations or Steps (202-214). These Operations are exemplary, and the executed method can omit one or more of the listed Operations, can repeat Operations, can include other Operations, or can execute the Operations concurrently, substantially simultaneously, or in another order, as appropriate or desired. The operations can be performed automatically by the processor or controller of a machine or computer, such as described below for FIG. 4.

At 202, a range for at least one setting of a laser included in a laser system can be determined using an artificial intelligence (AI) or machine learning (ML) or other algorithm (collectively, “the algorithm”). The operation of the algorithm is discussed below for FIG. 4. The range can be determined by the algorithm before a laser procedure such as a lithotripsy procedure such as by information about the medical procedure being entered by a physician on a user interface, or by retrieving the information about the medical procedure (e.g., what the procedure to be performed is, what devices will be used during the procedure, or the like) and/or patient information from a database connected to the computer on which the algorithm is being run or operated. For example, the range can be tied to or based on a particular type of medical procedure to be performed on a patient (e.g., kidney stone ablation), which can be retrieved from a patient file stored in the database. In another example, the physician can indicate the type of medical procedure, such as on a user interface, and the range can be retrieved from a file in the database with information specific to the particular medical procedure and without any patient-specific information or data. Once the algorithm is provided information about the procedure type, which may include information such as the composition of the kidney stone, the location of the kidney stone within the urinary tract system of the patient, the type of laser or lasers to be used during the procedure, or any additional relevant information, the algorithm can determine appropriate settings for the lasers. For example, the algorithm can determine an upper and lower limit of the range for laser intensity that is appropriate for the particular procedure, an upper and lower limit for a duty cycle at which to pulse the laser, or the like. The range can be a range of values chosen or determined by the physician performing the procedure or may be a set range for the procedure that is specific to the hospital or medical facility at which the procedure is being performed. The range can be edited or adjusted by the physician as desired, such as through a prompt or menu on the GUI.

At 204, the system can determine that the laser can operate automatically within the range. In an example, when the information about the medical procedure is retrieved, and the algorithm determines a range for the settings of a laser that are appropriate for the procedure, the algorithm can determine, based on the actual lasers being used during the procedure (e.g., a red-light laser, a blue-light laser, a green-light laser, or a combination of different types of lasers), that the lasers can operate within the determined range. The range and the determination that the particular lasers can operate within the determined range can also be determined by the physician performing the medical procedure, based on his prior experience on the operation. Thus, the algorithm can adjust the determined range if the laser is only partially capable of operating automatically within the determined range (e.g., if the laser can emit laser radiations at a lower limit of the determined range but not at an upper limit of the determined range). Additionally, or alternatively, the system can recommend a different laser be used during the procedure.

At 206, the algorithm can determine a need to adjust the at least one setting. This determination can be based on one or more factors or conditions that occur or are encountered during the medical procedure. In an example, the condition may be an environmental condition, such as a temperature and/or pressure at the target site in proximity to the tip of a scope being used during the procedure measured by a temperature sensor and/or pressure sensor coupled to the laser or scope. In another example, the need to adjust the setting can be based on a change in a characteristic of a target. For example, when the target is a kidney stone, the algorithm can determine, based on a change in size of the stone (determined by, for example, an image analysis of the stone) as it is being ablated that less laser radiation is needed to continue to break apart the stone. In another example, the system can determine a composition of the stone has changed (based on, for example, a spectroscopic analysis of signals reflected or emitted from the stone) and adjust the laser intensity or other setting(s) based on the change. For example, the composition of a stone may change from a harder substance to a softer substance or vice-versa. A stone may be comprised partially of Calcium Oxalate and Calcium Phosphate such that it has a hard “shell”, and a softer “core”, and different levels of laser radiation may be appropriate to ablate the different material types. The algorithm can determine, for example, based on spectral analysis of the stone by a spectrometer included or attached to the laser system described above for FIG. 1, that the composition of the stone has changed and adjust the setting based on that change.

In another example, the need to adjust the setting can be based on a distance of the laser fiber or the tip of the scope to the target, or a position of laser fiber or tip of the scope within the body of a patient. For example, the closer the laser fiber is to a kidney stone, the less energy may be required to ablate the stone. In another example, if the laser fiber is positioned such that it could emit laser radiation toward non-target tissue that could be affected by the radiation, the radiation or laser intensity can be lowered, or emission of the laser stopped, until the laser fiber and/or the scope is repositioned.

At 208, an indication can be provided on a user interface of an updated value of the at least one setting. For example, when the algorithm determines a need to adjust the setting at Operation 206, the system can notify the physician such as on a GUI that the laser setting needs to be adjusted and to what value the new setting will be changed. Additionally, the indication can include displaying a reason for the determined need. At 210, the laser emission can be disabled, locked, terminated, switched off, or otherwise prevented. This may occur contemporaneously with or shortly after the indication is provided at Operation 208. In another example, the locking of the laser can occur after the physician accepts the changed value after the indication or notification is provided to the physician. Or, stated differently, the laser can only be pulsed after the physician accepts the changed setting. At 212, the at least one setting can be adjusted to the updated value. The adjustment can be made while the emission of the laser is disabled at Operation 210. Adjusting the laser settings while the laser is disabled can provide a measure of safety, especially when the adjustment includes an increase in radiation intensity, as it can allow for the scope to be positioned to prevent stray radiation energy from contacting non-target tissue. At 214, laser emission can be resumed after adjustment of the at least one setting.

In some examples, more than one laser may be available to be used during the procedure. The Operations listed in the method 200, as well as all of the components discussed above for FIG. 1 can apply to a multiple laser system. In an example in which more than one laser is included, the adjustment can include switching from one laser to another, for example switching from a first laser to a second laser, based on any of the factors discussed above. Hence, the system 100 or the algorithm can selectively switch from a first laser to a second laser based on a change in an environmental condition, a change in location of the scope (e.g., the portion of anatomy in which the scope is located), a change in a characteristic of a target, a change in the medical procedure, or any factor or parameter warranting or necessitating a change from one type of laser to another. For example, when the medical procedure requires ablation of a stone and also requires cutting or cauterizing tissue, a first laser may be better suited for ablating the stone and the other better suited for cutting or cauterizing tissue. Thus, depending on what portion of the procedure is being performed, the laser actively emitting radiation can be changed or switched.

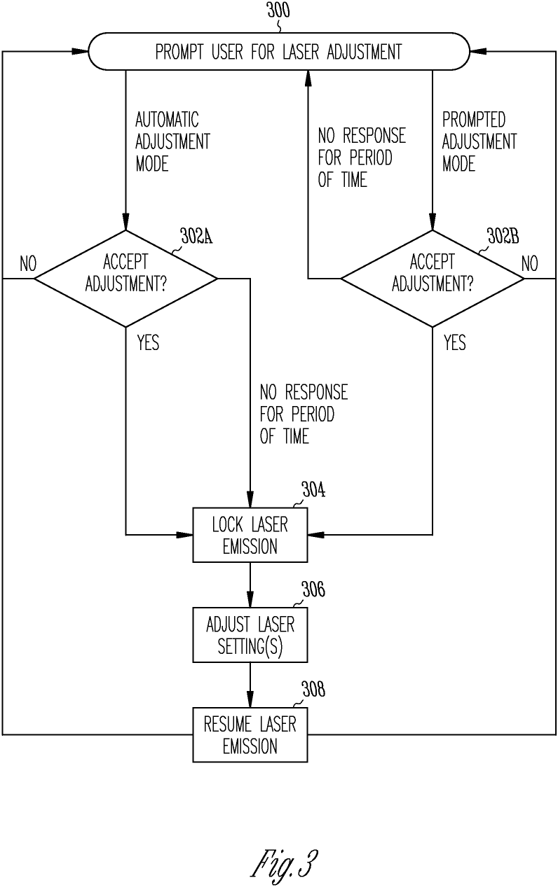

FIG. 3 is a flowchart for operation of a laser adjustment system. As illustrated in FIG. 3, at Operation 300, the laser system can prompt the user for laser adjustment. The laser system can prompt the user after a determination that one or more settings of the laser system should be adjusted according to the methods described above (such as in the method illustrated and discussed for FIG. 2, above. When the laser system is operating in an automatic adjustment mode, at Operation 303, a determination can be made at 302A whether to accept, reject, or ignore the prompt for adjustment of the laser settings. For example, the laser system may use a pop-up or pop-out screen on a GUI to notify the user that an adjustment to the one or more settings is recommended or necessary and may prompt the user to accept or decline the settings change, such as through a button on the pop-up screen that the user can select to accept the proposed change(s) and/or a button that the user can select to cancel or reject the proposed change(s).

If, at 302A, the user cancels or rejects the changes, such as by selecting a “cancel” or “reject” option on the pop-up screen, the laser system can resume using the current laser setting(s) until such time a new adjustment is proposed at which point the laser system can return to Operation 300. If at 302, the user accepts the proposed change(s), the laser system can proceed to Operation 304 and lock or disable the laser emission, adjust the one or more laser settings at Operation 306, and resume operation or emission of the laser with the updated setting(s) at Operation 308, at which point, when another adjustment is required, the laser system can return to Operation 300. If, however, at 302A the user does not respond to the indication (e.g., the user does not affirmatively accept the adjustment, but does not reject the adjustment within a period of time, such as within five seconds, or any appropriate or desired amount of time) the laser system can automatically proceed to Operations 304, 306, and 308, and disable the laser emission, adjust the setting(s) and resume laser emission without additional user interaction as long as the user continues to engage laser emission (e.g., as long as a footswitch continues to be depressed a button or trigger is pressed, or any actuation member to activate the laser remains engaged).

When the laser system is operating in a prompted adjustment mode, at Operation 302B, the user can decide whether to accept, reject, or ignore the prompt sent at Operation 300. If at 302B, the user rejects the proposed change(s) or does not accept or reject the proposed changes within a period of time (e.g., five seconds), the laser system will continue to operate with the current laser setting(s) and return to Operation 300 when a need for another adjustment is required. In the prompted adjustment mode it is only when the user affirmatively accepts the proposed change(s) to the laser settings that the laser system can continue to Operations 304, 306, and 308 to update the settings and continue laser emission with the updated settings.

While the examples illustrated in FIGS. 2 and 3 show the laser emission being locked or disabled after the indication and prompt to accept or reject the proposed change(s) to the setting(s) are made, the laser system can disable or lock the laser emission before, or contemporaneously with the prompt displayed at Operation 300. Furthermore, the laser system can be switched from automatic adjustment mode to prompted adjustment mode, such as during the medical procedure by the user as desired. Additionally, or alternatively, the laser system can automatically switch between prompted adjustment mode and automatic adjustment mode such as based on what part of the medical procedure is being performed. For example, a user may desire to have more control over the laser system during certain parts of the procedure and the laser system can be programmed or set before the procedure to remain in prompted adjustment mode during those parts of the procedure and then switch to automatic adjustment mode during other parts of the procedure in which the user deems automatic adjustment mode to be acceptable.

FIG. 4 is a block diagram of an example of a machine 400 upon which any one or more of the techniques (e.g., methodologies) discussed herein may perform. The machine 400 may operate as a standalone device or may be connected (e.g., networked) to other machines. For example, the machine 400 can be included in or connected to the surgical laser 102 and/or the scope 124 and may include components such as the graphical user interface 104, the processor 112, the controller 114, or the surgical fiber actuator 122, discussed above. Additionally, or alternatively, the machine 400 may operate the algorithm discussed above for FIG. 2, or the computer-based clinical decision support system (CDSS) discussed below for FIG. 4. In a networked deployment, the machine 400 may operate in the capacity of a server machine, a client machine, or both in server-client network environments. In an example, the machine 400 may act as a peer machine in peer-to-peer (P2P) (or other distributed) network environment. The machine 400 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine 400 (e.g., computer system) may include a hardware processor 402 (e.g., a central processing unit (CPU), a graphics processing unit (GPU), a hardware processor core, field programmable gate array (FPGA), or any combination thereof), a main memory 404 and a static memory 406, some or all of which may communicate with each other via an interlink (e.g., bus) 430. The machine 400 may further include a display unit 410, an alphanumeric input device 412 (e.g., a keyboard), and a user interface (UI) navigation device 414 (e.g., a mouse). In an example, the display unit 410, input device 412 and UI navigation device 414 may be a touch screen display. The machine 400 may additionally include a storage device 408 (e.g., drive unit), a signal generation device 418 (e.g., a speaker), a network interface device 420, and one or more sensors 416, such as a global positioning system (GPS) sensor, compass, accelerometer, or other sensor. The machine 400 may include an output controller 428, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 408 may include a machine readable medium 422 (e.g., a non-transitory medium) on which is stored one or more sets of data structures or instructions 424 (e.g., software) embodying or used by any one or more of the techniques or functions described herein. The instructions 424 may also reside, completely or at least partially, within the main memory 404, within static memory 406, or within the hardware processor 402 during execution thereof by the machine 400. In an example, one or any combination of the hardware processor 402, the main memory 404, the static memory 406, or the storage device 408 may constitute machine readable media.

While the machine readable medium 422 is illustrated as a single medium, the term “machine readable medium” may include a single medium or multiple media (e.g., a centralized or distributed database, and/or associated caches and servers) configured to store the one or more instructions 424. The term “machine readable medium” may include any non-transitory medium that is capable of storing, encoding, or carrying instructions for execution by the machine 400 and that cause the machine 400 to perform any one or more of the techniques of the present disclosure, or that is capable of storing, encoding, or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

FIG. 5 shows a schematic diagram of an exemplary computer-based clinical decision support system (CDSS) 500 that is configured to implement or recommend changed laser settings based on information about a target being treated, a piece or portion of tissue being treated, or conditions encountered during a laser medical procedure. In various embodiments, the CDSS 500 includes an input interface 502 through which one or more laser setting ranges, which are specific to a patient, are provided as input features to an artificial intelligence (AI) model 504, a processor, such as processor 402, which performs an inference operation in which the information regarding the target or a piece or portion of tissue are applied to the AI model to generate the one or more changed laser settings, and a user interface (UI) through which one or more changed laser settings is communicated to a user, e.g., a clinician.

In some embodiments, the input interface 502 may be a direct data link between the CDSS 500 and one or more medical devices that generate at least some of the input features. For example, the input interface 502 may transmit information about the target, such as composition or density of the target, directly to the CDSS 500 during a therapeutic and/or diagnostic medical procedure. Additionally, or alternatively, the input interface 502 may be a classical user interface that facilitates interaction between a user and the CDSS 500. For example, the input interface 502 may facilitate a user interface through which the user may manually enter a range of laser settings that the laser system can propose during the procedure that are specific to the patient. Additionally, or alternatively, the input interface 502 may provide the CDSS 500 with access to an electronic patient record from which one or more input features may be extracted. In any of these cases, the input interface 502 is configured to collect one or more of the following input features in association with a specific patient on or before a time at which the CDSS 500 is used to assess:
Information regarding target characteristics 510, which can include information regarding the composition of the target, information regarding the density of the target, or information regarding the size of the target;
Information regarding a change in the target characteristics 512 (e.g., a change in composition or a change in density of the target);
An environmental condition;
A location of the laser fiber and/or scope;
Information about the medical procedure;
An indication of non-target tissue; and/or
An allowable range of laser settings specific to the patient or the procedure being performed.

Based on one or more of the above input features, the processor 402 performs an inference operation using the AI model to generate a change in one or more laser settings to be implemented or a recommended change in one or more laser settings to be proposed to the user. For example, input interface 502 may deliver the target characteristics, determined changes in the target characteristics, and the range of permissible laser settings into an input layer of the AI model which propagates these input features through the AI model to an output layer. The AI model can provide a computer system the ability to perform tasks, without explicitly being programmed, by making inferences based on patterns found in the analysis of data. The AI model explores the study and construction of algorithms (e.g., machine-learning algorithms) that may learn from existing data and make predictions about new data. Such algorithms operate by building an AI model from example training data in order to make data-driven predictions or decisions expressed as outputs or assessments.

There are two common modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model can implement the same relationships when given inputs to generate the corresponding outputs. Unsupervised ML is the training of an ML algorithm using information that is neither classified nor labeled and allowing the algorithm to act on that information without guidance. Unsupervised ML is useful in exploratory analysis because it can automatically identify structure in data.

Certain tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM).

Some tasks for unsupervised ML include clustering, representation learning, and density estimation. Some examples of unsupervised-ML algorithms are K-means clustering, principal component analysis, and autoencoders.

Another type of ML is federated learning (also referred to as collaborative learning) that trains an algorithm across multiple decentralized devices holding local data, without exchanging the data. This approach stands in contrast to centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing to address critical issues such as data privacy, data security, data access rights and access to heterogeneous data.

In some examples, the AI model may be trained continuously or periodically prior to performance of the inference operation by the processor 402. Then, during the inference operation, the patient specific input features provided to the AI model may be propagated from an input layer, through one or more hidden layers, and ultimately to an output layer that corresponds to the changes to one or more of the laser settings. For example, when the target is a kidney stone, based on the characteristics of the stone and a determined change in the target characteristics (e.g., a change in composition of the stone), a change in the laser settings such as laser intensity and/or frequency can be propagated to the output layer.

During and/or subsequent to the inference operation, the change in the laser settings may be communicated to the user via the user interface (UI) and/or automatically cause the processor 402 to automatically adjust the laser settings and proceed with the laser procedure using the new settings.

### ADDITIONAL NOTES AND EXAMPLES

Example 1 is a laser system comprising: a processor; a user interface coupled to the processor; and memory coupled to the processor, the memory configured to store instructions that, when executed by the processor, cause the processor to: receive a range for at least one setting of a first laser of the laser system that the first laser can operate within; determine a proposed updated value of the at least one setting; determine whether the proposed updated value is within the received range; provide, via the user interface, (i) an indication of the proposed updated value and (ii) an option for a user to accept or reject the proposed updated value for the at least one setting; and responsive to an acceptance of the proposed updated value or no rejection of the proposed updated value by the user within a predetermined duration, adjust the at least one setting based on the proposed updated value.

In Example 2, the subject matter of Example 1 optionally includes wherein the instructions cause the processor to: temporarily disable the first laser after receiving an acceptance or no rejection of the proposed updated value by the user.

In Example 3, the subject matter of any one or more of Examples 1-2 optionally include wherein the instructions cause the processor to: temporarily disable emission of the first laser during indicating the proposed updated value until at least one of adjustment of the at least one setting based on the proposed updated value or user rejection of the proposed updated value.

In Example 4, the subject matter of any one or more of Examples 1-3 optionally include wherein the instructions cause the processor to: determine a characteristic of a target at a first time; determine a change in the characteristic of the target at a second time; and determine the proposed updated value of the at least one setting based on the change in the characteristic of the target at the second time.

In Example 5, the subject matter of any one or more of Examples 1-4 optionally include wherein the instructions cause the processor to: determine the proposed updated value of the at least one setting based on at least one of a distance of a laser fiber from a target or a position of the laser fiber within a portion of anatomy that at least one of a scope or a laser fiber connected to the laser system is within.

In Example 6, the subject matter of any one or more of Examples 1-5 optionally include wherein the instructions cause the processor to: determine the proposed updated value of the at least one setting based on an environmental condition measured by a sensor coupled to the processor.

In Example 7, the subject matter of Example 6 optionally includes wherein the sensor includes one or more of a temperature sensor, a pressure sensor, or an accelerometer.

In Example 8, the subject matter of any one or more of Examples 1-7 optionally include wherein the at least one setting includes an amount of energy emitted by the first laser, wherein the at least one setting includes one or more of an intensity of laser light or laser radiation of the first laser, a wavelength of the first laser, a pulse width of the first laser, or a duty cycle of the first laser.

In Example 9, the subject matter of any one or more of Examples 1-8 optionally include a feedback mechanism provides an additional indication, wherein the additional indication provides a warning to the user, and wherein the feedback mechanism includes one or more of a haptic feedback mechanism, an illumination feedback mechanism, or an audible feedback mechanism.

In Example 10, the subject matter of Example 9 optionally includes wherein the warning to the user warns the user that at least one of: the at least one setting of the first laser is within a threshold amount of an upper limit of the range or the proposed updated value has been determined.

In Example 11, the subject matter of any one or more of Examples 9-10 optionally include wherein the illumination feedback mechanism includes changing a characteristic of a user-visible targeting illumination beam emitted toward a target.

In Example 12, the subject matter of any one or more of Examples 1-11 optionally include wherein the acceptance of the proposed updated value of is initiated through a voice command or an activation of an actuation member that includes a button or switch on a handpiece connected to the laser system, or a foot switch connected to the laser system.

In Example 13, the subject matter of any one or more of Examples 1-12 optionally include wherein at least one of the range or a limit of the range is determined based on at least one of a portion of anatomy that at least one of a scope or a laser fiber connected to the laser system is within, a type of laser fiber included in the laser system, or a type of medical procedure for which the laser system is being used.

In Example 14, the subject matter of Example 13 optionally includes wherein the portion of anatomy is determined based on an image recognition technique performed on an image from an imaging device connected to the scope.

In Example 15, the subject matter of any one or more of Examples 13-14 optionally include wherein the laser system is communicatively coupled to a database containing patient information, wherein the type of medical procedure is determined from the patient information in the database.

In Example 16, the subject matter of any one or more of Examples 1-15 optionally include wherein the laser system includes a second laser of a different type than the first laser, and wherein the instructions cause the processor to: receive a second range for at least one setting of the second laser that the second laser can operate within; determine a second proposed updated value of the at least one setting of the second laser; determine whether the second proposed updated value is within the received second range; provide, via the user interface, (iii) an indication of the second proposed updated value and (iv) an option to accept or reject the second proposed updated value for the at least one setting one setting of the second laser; responsive to an acceptance of the second proposed updated value of the at least one setting of the second laser, adjust the at least one setting of the second laser based on the second proposed updated value of the at least one setting of the second laser; select one of the first laser or the second laser based on at least one of an environmental condition measured by a sensor coupled to the processor, a portion of anatomy at least one of a laser fiber or a scope connected to the laser system is within, or a type of medical procedure for which the laser system is being used; and selectively switch to the other of the first laser or the second laser based on a change in the environmental condition, a change in the portion of anatomy, or a change in the medical procedure.

In Example 17, the subject matter of Example 16 optionally includes wherein responsive to no rejection of the second proposed updated value of the at least one setting of the second laser within a predetermined duration, adjust the at least one setting of the second laser based on the second proposed updated value of the at least one setting of the second laser.

In Example 18, the subject matter of any one or more of Examples 16-17 optionally include wherein responsive to no rejection of the second proposed updated value of the at least one setting of the second laser within a predetermined duration, cancel an adjustment to the at least one setting of the second laser.

Example 19 is a computer implemented method for automatic adjustment of a laser system, the method comprising: determining, using a computer implemented processor, a range for at least one setting of a laser included in the laser system; determining, using the computer implemented processor, a need to adjust the at least one setting; providing an indication on a user interface of an updated value of the at least one setting; verifying, using the computer implemented processor, whether the updated value is within the range; disabling, via controller circuitry of a computer, emission of the laser; adjusting, via the controller circuitry, the at least one setting of the laser to the updated value of the at least one setting while the laser is disabled; and resuming emission of the laser after adjustment of the at least one setting of the laser.

In Example 20, the subject matter of Example 19 optionally includes wherein determining the need to adjust the at least one setting is based on at least one of a characteristic of a target, a change in the characteristic of the target, a distance of a laser fiber from the target, a position of the laser fiber, or an environmental condition measured by a sensor coupled to the laser system, and wherein an upper limit of the range is determined based on at least one of a portion of anatomy a scope connected to the laser system is within, a type of laser fiber included in the laser system, or a medical procedure for which the laser system is being used.

Example 21 is a laser system comprising: a processor; a user interface coupled to the processor; and memory coupled to the processor, the memory configured to store instructions that, when executed by the processor, cause the processor to: receive a first range for at least one setting of a first laser of the laser system that the first laser can automatically adjust within; receive a second range for at least one setting of a second laser of the laser system that the second laser can automatically adjust within; determine at least one of a proposed updated value for the at least one setting of the first laser or a second proposed updated value for the at least one setting of the second laser; determine whether the proposed updated value for the at least one setting of the first laser is within the first range; determine whether the second proposed updated value for the at least one setting of the second laser is within the second range; provide, via the user interface, (i) an indication of the at least one of the proposed updated value for the at least one setting of the first laser or the second proposed updated value for the at least one setting of the second laser and (ii) an option to accept or reject the at least one of the proposed updated value for the at least one setting of the first laser or the second proposed updated value for the at least one setting of the second laser; and responsive to an acceptance of at least one of the proposed updated value or the second proposed updated value, adjust at least one of the at least one setting of the first laser or the at least one setting of the second laser.

In Example 22, the subject matter of Example 21 optionally includes wherein responsive to no rejection of the at least one of the proposed updated value or the second proposed updated value by the user within a predetermined period of time, the instructions cause the processor to: adjust at least one of the at least one setting of the first laser or the at least one setting of the second laser.

In Example 23, the subject matter of any one or more of Examples 21-22 optionally include wherein responsive to no rejection of the at least one of the proposed updated value or the second proposed updated value by the user within a predetermined period of time, the instructions cause the processor to: cancel adjustment of at least one of the at least one setting of the first laser or the at least one setting of the second laser.

In Example 24, the subject matter of any one or more of Examples 21-23 optionally include wherein the proposed updated value for the at least one setting of the first laser is within the first range, wherein the second proposed updated value for the at least one setting of the second laser is within the second range, and wherein the instructions cause the processor to: select one of the first laser or the second laser based on at least one of an environmental condition measured by a sensor coupled to the processor, a portion of anatomy a scope connected to the laser system is within, or a medical procedure for which the laser system is being used; and selectively switch to the other of the first laser or the second laser based on a change in the environmental condition, a change in the portion of anatomy, or a change in the medical procedure.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A laser system comprising:
a processor;
a user interface coupled to the processor; and
memory coupled to the processor, the memory configured to store instructions that, when executed by the processor, cause the processor to:
receive a range for at least one setting of a first laser of the laser system that the first laser can operate within;
determine a proposed updated value of the at least one setting;
determine whether the proposed updated value is within the received range;
provide, via the user interface, (i) an indication of the proposed updated value and (ii) an option for a user to accept or reject the proposed updated value for the at least one setting; and
responsive to an acceptance of the proposed updated value or no rejection of the proposed updated value by the user within a predetermined duration, adjust the at least one setting based on the proposed updated value.

2. The laser system of claim 1, wherein the instructions cause the processor to:
temporarily disable the first laser after receiving an acceptance or no rejection of the proposed updated value by the user.

3. The laser system of claim 1, wherein the instructions cause the processor to:
temporarily disable emission of the first laser during indicating the proposed updated value until at least one of adjustment of the at least one setting based on the proposed updated value or user rejection of the proposed updated value.

4. The laser system of claim 1, wherein the instructions cause the processor to:
determine a characteristic of a target at a first time;
determine a change in the characteristic of the target at a second time; and
determine the proposed updated value of the at least one setting based on the change in the characteristic of the target at the second time.

5. The laser system of claim 1, wherein the instructions cause the processor to:
determine the proposed updated value of the at least one setting based on at least one of a distance of a laser fiber from a target or a position of the laser fiber within a portion of anatomy that at least one of a scope or a laser fiber connected to the laser system is within.

6. The laser system of claim 1, wherein the instructions cause the processor to:
determine the proposed updated value of the at least one setting based on an environmental condition measured by a sensor coupled to the processor.

7. The laser system of claim 6, wherein the sensor includes one or more of a temperature sensor, a pressure sensor, or an accelerometer.

8. The laser system of claim 1, wherein the at least one setting includes an amount of energy emitted by the first laser, wherein the at least one setting includes one or more of an intensity of laser light or laser radiation of the first laser, a wavelength of the first laser, a pulse width of the first laser, or a duty cycle of the first laser.

9. The laser system of claim 1, comprising:
a feedback mechanism provides an additional indication, wherein the additional indication provides a warning to the user, and wherein the feedback mechanism includes one or more of a haptic feedback mechanism, an illumination feedback mechanism, or an audible feedback mechanism.

10. The laser system of claim 9, wherein the warning to the user warns the user that at least one of: the at least one setting of the first laser is within a threshold amount of an upper limit of the range or the proposed updated value has been determined.

11. The laser system of claim 9, wherein the illumination feedback mechanism includes changing a characteristic of a user-visible targeting illumination beam emitted toward a target.

12. The laser system of claim 1, wherein the acceptance of the proposed updated value of is initiated through a voice command or an activation of an actuation member that includes a button or switch on a handpiece connected to the laser system, or a foot switch connected to the laser system.

13. The laser system of claim 1, wherein at least one of the range or a limit of the range is determined based on at least one of a portion of anatomy that at least one of a scope or a laser fiber connected to the laser system is within, a type of laser fiber included in the laser system, or a type of medical procedure for which the laser system is being used.

14. The laser system of claim 13, wherein the portion of anatomy is determined based on an image recognition technique performed on an image from an imaging device connected to the scope.

15. The laser system of claim 13, wherein the laser system is communicatively coupled to a database containing patient information, wherein the type of medical procedure is determined from the patient information in the database.

16. The laser system of claim 1, wherein the laser system includes a second laser of a different type than the first laser, and wherein the instructions cause the processor to:
receive a second range for at least one setting of the second laser that the second laser can operate within;
determine a second proposed updated value of the at least one setting of the second laser;
determine whether the second proposed updated value is within the received second range;
provide, via the user interface, (iii) an indication of the second proposed updated value and (iv) an option to accept or reject the second proposed updated value for the at least one setting one setting of the second laser;
responsive to an acceptance of the second proposed updated value of the at least one setting of the second laser, adjust the at least one setting of the second laser based on the second proposed updated value of the at least one setting of the second laser;
select one of the first laser or the second laser based on at least one of an environmental condition measured by a sensor coupled to the processor, a portion of anatomy at least one of a laser fiber or a scope connected to the laser system is within, or a type of medical procedure for which the laser system is being used; and
selectively switch to the other of the first laser or the second laser based on a change in the environmental condition, a change in the portion of anatomy, or a change in the medical procedure.

17. The system of claim 16, wherein responsive to no rejection of the second proposed updated value of the at least one setting of the second laser within a predetermined duration, adjust the at least one setting of the second laser based on the second proposed updated value of the at least one setting of the second laser.

18. The system of claim 16, wherein responsive to no rejection of the second proposed updated value of the at least one setting of the second laser within a predetermined duration, cancel an adjustment to the at least one setting of the second laser.

19. A computer implemented method for automatic adjustment of a laser system, the method comprising:
determining, using a computer implemented processor, a range for at least one setting of a laser included in the laser system;
determining, using the computer implemented processor, a need to adjust the at least one setting;
providing an indication on a user interface of an updated value of the at least one setting;
verifying, using the computer implemented processor, whether the updated value is within the range;
disabling, via controller circuitry of a computer, emission of the laser;
adjusting, via the controller circuitry, the at least one setting of the laser to the updated value of the at least one setting while the laser is disabled; and
resuming emission of the laser after adjustment of the at least one setting of the laser.

20. The method of claim 19, wherein determining the need to adjust the at least one setting is based on at least one of a characteristic of a target, a change in the characteristic of the target, a distance of a laser fiber from the target, a position of the laser fiber, or an environmental condition measured by a sensor coupled to the laser system, and wherein an upper limit of the range is determined based on at least one of a portion of anatomy a scope connected to the laser system is within, a type of laser fiber included in the laser system, or a medical procedure for which the laser system is being used.

21. A laser system comprising:
a processor;
a user interface coupled to the processor; and
memory coupled to the processor, the memory configured to store instructions that, when executed by the processor, cause the processor to:
receive a first range for at least one setting of a first laser of the laser system that the first laser can automatically adjust within;
receive a second range for at least one setting of a second laser of the laser system that the second laser can automatically adjust within;
determine at least one of a proposed updated value for the at least one setting of the first laser or a second proposed updated value for the at least one setting of the second laser;
determine whether the proposed updated value for the at least one setting of the first laser is within the first range;
determine whether the second proposed updated value for the at least one setting of the second laser is within the second range;
provide, via the user interface, (i) an indication of the at least one of the proposed updated value for the at least one setting of the first laser or the second proposed updated value for the at least one setting of the second laser and (ii) an option to accept or reject the at least one of the proposed updated value for the at least one setting of the first laser or the second proposed updated value for the at least one setting of the second laser; and
responsive to an acceptance of at least one of the proposed updated value or the second proposed updated value, adjust at least one of the at least one setting of the first laser or the at least one setting of the second laser.

22. The laser system of claim 21, wherein responsive to no rejection of the at least one of the proposed updated value or the second proposed updated value by the user within a predetermined period of time, the instructions cause the processor to:
adjust at least one of the at least one setting of the first laser or the at least one setting of the second laser.

23. The laser system of claim 21, wherein responsive to no rejection of the at least one of the proposed updated value or the second proposed updated value by the user within a predetermined period of time, the instructions cause the processor to:
cancel adjustment of at least one of the at least one setting of the first laser or the at least one setting of the second laser.

24. The laser system of claim 21, wherein the proposed updated value for the at least one setting of the first laser is within the first range, wherein the second proposed updated value for the at least one setting of the second laser is within the second range, and wherein the instructions cause the processor to:
select one of the first laser or the second laser based on at least one of an environmental condition measured by a sensor coupled to the processor, a portion of anatomy a scope connected to the laser system is within, or a medical procedure for which the laser system is being used; and
selectively switch to the other of the first laser or the second laser based on a change in the environmental condition, a change in the portion of anatomy, or a change in the medical procedure.
